(19) United States
(12) Patent Application Publication
(10) Pub. No.: US 2007/0087324 A1
(43) Pub. Date: Apr. 19, 2007

(54) METHOD OF PROVIDING READILY AVAILABLE CELLULAR MATERIAL DERIVED FROM PERIPHERAL BLOOD

(76) Inventor: Donnie RUDD, Sugar Land, TX (US)

Correspondence Address: LADAS & PARRY LLP, 224 SOUTH MICHIGAN AVENUE, SUITE 1600, CHICAGO, IL, 60604, US

(21) Appl. No.: 11/565,021

(22) Filed: Nov. 30, 2006

Related U.S. Application Data
(62) Division of application No. 11/363,702, filed on Feb. 27, 2006.
(60) Provisional application No. 60/657,271, filed on Feb. 28, 2005.
(60) Provisional application No. 60/657,306, filed on Feb. 28, 2005.

(51) Int. Cl. A01N 1/02; C12N 5/08

(52) U.S. Cl. 435/2; 435/372

(57) ABSTRACT

Process for preparing a peripheral blood cell composition in a TVEMF-bioreactor.

### CROSS-REFERENCE TO RELATED APPLICATIONS

This is a Divisional Application, the parent patent application being Ser. No. 11/363,702 filed on Feb. 27, 2006. The entire declaration, oath, specification, disclosure, and drawing figures, and each of them, from said parent patent application are hereby incorporated herein by reference, thereto.

### FIELD OF THE INVENTION

The present invention is directed to a process for preparing adult stem cells from peripheral blood in a TVEMF-bioreactor.

### BACKGROUND OF THE INVENTION

Regeneration of mammalian, particularly human, tissue has long been a desire of the medical community. Thus far, repair of human tissue has been accomplished largely by transplantations of like tissue from a donor. Beginning essentially with the kidney transplant from one of the Herrick twins to the other and later made world famous by South African Doctor Christian Barnard's transplant of a heart from Denise Darval to Louis Washkansky on Dec. 3, 1967, tissue transplantation became a widely accepted method of extending life in terminal patients.

Transplantation of human tissue, from its first use, encountered major problems, primarily tissue rejection due to the body's natural immune system. This often caused the use of tissue transplantation to have a limited prolongation of life (Washkansky lived only 18 days past the surgery).

In order to overcome the problem of the body's immune system, numerous anti-rejection drugs (e.g. Imuran, Cyclosporine) were soon developed to suppress the immune system and thus prolong the use of the tissue prior to rejection. However, the rejection problem has continued creating the need for an alternative to tissue transplantation.

Bone marrow transplantation has also been used, and is still the procedure of choice for treatment of some illnesses, such as leukemia, to repair certain tissues such as bone marrow, but bone marrow transplantation also has problems. It requires a match from a donor (found less than 50% of the time); it is painful, expensive, and risky. Consequently, an alternative to bone marrow transplantation is highly desirable. Transplantation of tissue stem cells such as the transplantation of liver stem cells found in U.S. Pat. No. 6,129,911 have similar limitations rendering their widespread use questionable.

In recent years, researchers have experimented with the use of pluripotent embryonic stem cells as an alternative to tissue transplant. The theory behind the use of emnbryonic stem cells has been that they can theoretically be utilized to regenerate virtually any tissue in the body. The use of embryonic stem cells for tissue regeneration, however, has also encountered problems. Among the more serious of these problems are that transplanted embryonic stem cells have limited controllability, they sometimes grow into tumors, and the human embryonic stem cells that are available for research would be rejected by a patient's immune system (Nature, Jun. 17, 2002: Pearson, “Stem Cell Hopes Double”, news@nature.com, published online: Jun. 12, 2002). Further, widespread use of embryonic stem cells is so burdened with ethical, moral, and political concerns that its widespread use remains questionable.

The pluripotent nature of stem cells was first discovered fromn an adult stem cell found in bone marrow. Verfaille, C. M. et al., Pluripotency of mesenchymal stem cells derived from adult marrow. Nature 417, published online 20 June; doi:10.1038/nature00900, (2002) cited by Pearson, H. Stem cell hopes double. news@nature.com, published online: Jun. 21, 2002; doi: 10. 1038/news020617-11.

Boyse et al., U.S. Pat. No. 6,569,427 B1, discloses the cryopreservation and usefulness of cryopreserved fetal or neonatal blood in the treatment or prevention of various diseases and disorders such as anemias, malignancies, autoimmune disorders, and various immune dysfunctions and deficiencies. Boyse also discloses the use of hematopoietic reconstitution in gene therapy with the use of a heterologous gene sequence. The Boyse disclosure stops short, however, of expansion of cells for therapeutic uses. CorCell, a cord blood bank, provides statistics on expansion, cryopreservation, and transplantation of umbilical cord blood stem cells. “Expansion of Umbilical Cord Blood Stem Cells”, Information Sheet Umbilical Cord Blood, CorCell, Inc. (2003). One expansion process discloses utilizing a bioreactor with a central collagen based matrix. Research Center Julich: Blood Stem Cells from the Bioreactor. Press release May 17, 2001.

Research continues in an effort to elucidate the molecular mechanisms involved in the expansion of stem cells. For example, the CorCell article discloses that a signal molecule named Delta-1 aids in the development of cord blood stem cells. Ohishi K. et al.: Delta-1 enhances marrow and thymus repopulating ability of human CD34+/CD38− cord blood cells. Clin. Invest. 110:1165-1174 (2002).

Throughout this application, the term “peripheral blood” means blood that circulates, or has circulated, systematically in a mammal. The term “peripheral blood cells” means cells found in peripheral blood.

While adult stem cells can be found in numerous mature tissues, they are found in lesser quantities and are harder to locate. Also, stem cells found in tissues may be dedicated to that tissue, and less able to function as a truly pluripotent cell. Peripheral blood cells, however, are more readily available than stem cells in tissues.

There is a need, therefore, to provide a method and process of repairing human tissue that is not based on organ transplantation, bone marrow transplantation, or embryonic stem cells, and yet provides a composition of expanded peripheral blood stem cells, preferably in a therapeutic condition and dosage and unlikely to elicit an immune response, for use in a matter of hours rather than days.

### SUMMARY OF THE INVENTION

The present invention relates to a process for preparing a peripheral blood stem cell composition comprising the stops of placing a peripheral blood mixture in a culture chamber of a TVEMF-bioreactor; and subjecting the peripheral blood mixture to a TVEMF and TVEMF-expanding the peripheral blood stem cells in the TVEMF-bioreactor to prepare the peripheral blood stem cell composition. Preferably, the TVEMF applied to the cells is from about 0.05 to about 6.0 gauss. The present invention also relates to a method of cryopreserving the expanded stem cells by lowering their temperature to −120° C. to −1960° C. for one year or longer, and raising the temperature thereafter to a temperature suitable for introducing the cells into a mammal.

It is particularly desirable to have cryogenically TVEMF-expanded cells according to the present invention available from birth or an early age forward, for instance in case of an emergency crisis where every minute in delaying treatment can mean the difference in life or death. Also, the use of peripheral blood in the present invention makes the present cells and compositions readily available (at least in view of the readily available nature of peripheral blood).

### BRIEF DESCRIPTION OF THE DRAWINGS

In the drawings,

FIG. 1 schematically illustrates a preferred embodiment of a culture carrier flow loop of a bioreactor;

FIG. 2 is an elevated side view of a preferred embodiment of a TVEMF-bioreactor of the invention;

FIG. 3 is a side perspective of a preferred embodiment of the TVEMF-biorcactor of FIG. 2;

FIG. 4 is a vertical cross sectional view of a preferred embodiment of a TVEMF-bioreactor;

FIG. 5 is a vertical cross sectional view of a TVEMF-bioreactor;

FIG. 6 is an elevated side view of a time varying eletromagnetic force device that can house, and provide a time varying electromagnetic force to, a bioreactor;

FIG. 7 is a front view of the device shown in FIG. 6; and

FIG. 8 is a front view of the device shown in FIG. 6, further showing a bioreactor therein.

### DETAILED DESCRIPTION OF THE DRAWINGS

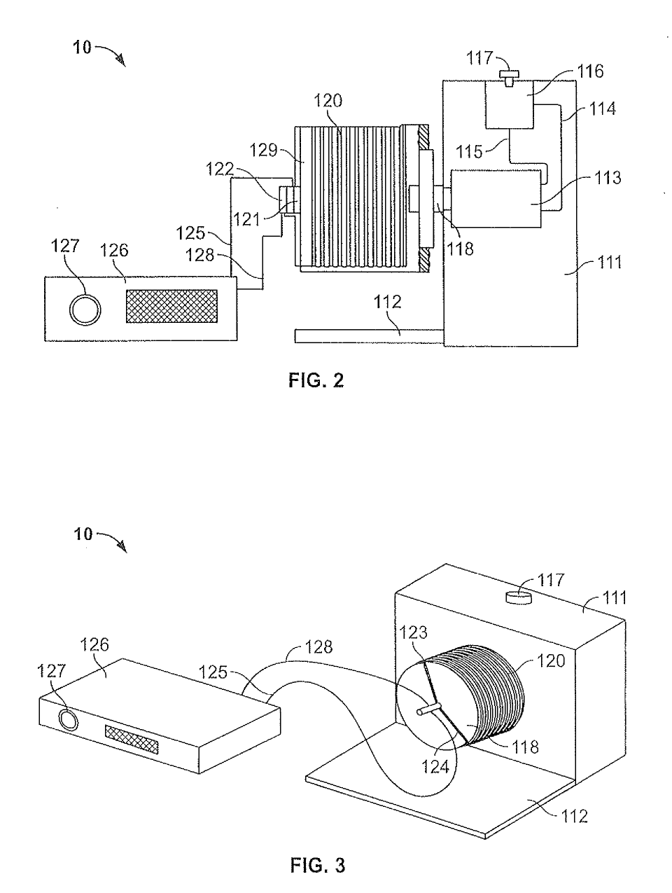

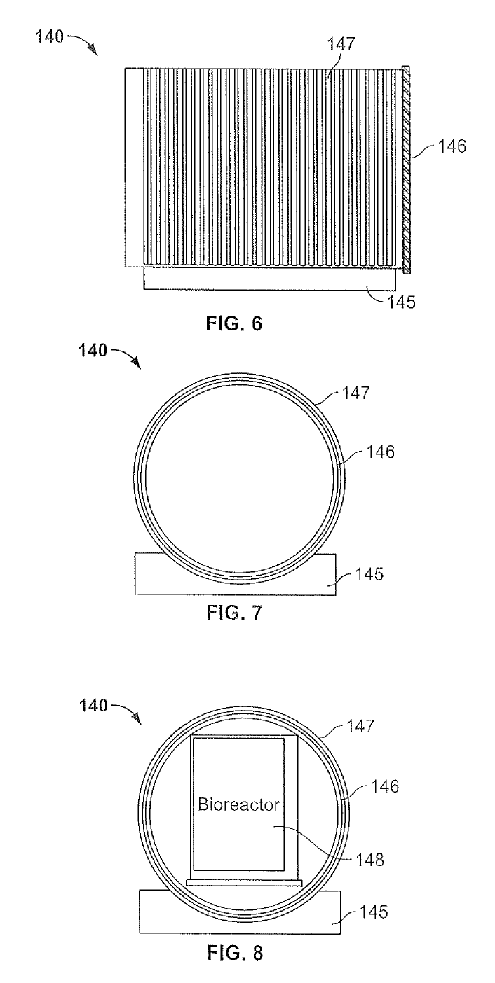

In the simplest terms, a rotating TVEMF-bioreactor comprises a cell culture chamber and a time varying electromagnetic force source. In operation, a peripheral blood mixture is placed into the cell culture chamber. The cell culture chamber is rotated over a period of time during which a time varying electromagnetic force is generated in the chamber by the time varying electromagnetic force source. Upon completion of the period of time, the TVEMF-expanded peripheral blood mixture is removed from the chamber. In a more complex TVEMF-bioreactor system, the time varying electromagnetic force source can be integral to the TVEMF-bioreactor, as illustrated in FIGS. 2-5, but can also be adjacent to a bioreactor as in FIGS. 6-8. Furthermore, a fluid carrier such as cell culture media or buffer (preferably similar to that media added to a peripheral blood mixture, discussed below), which provides sustenance to the cells. can be periodically refreshed and removed. Preferred TVEMF-bioreactors are described herein.

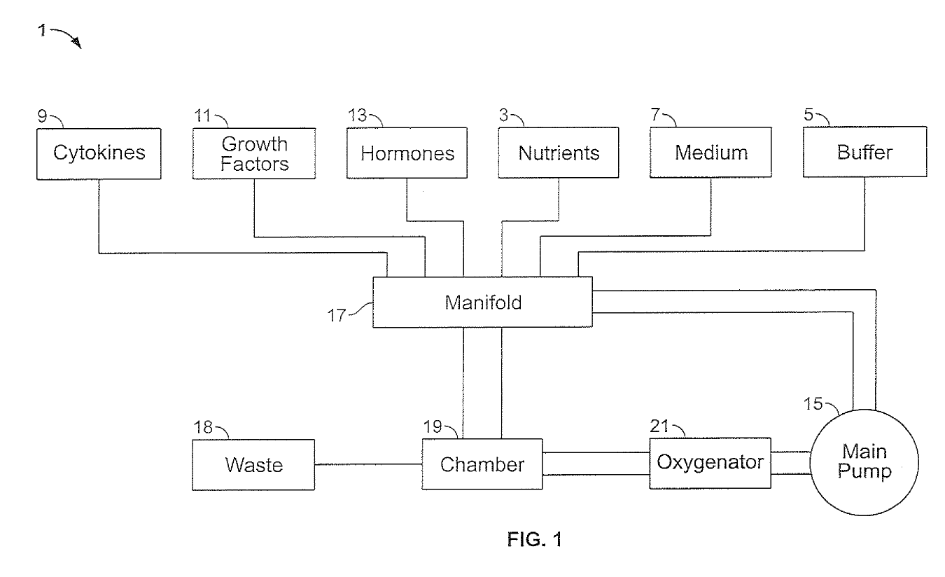

Referring now to FIG. 1, illustrated is a preferred embodiment of a culture carrier flow loop 1 in an overall bioreactor culture system for growing mammalian cells having a cell culture chamber 19, preferably a rotating cell culture chamber, an oxygenator 21, an apparatus for facilitating the directional flow of the culture carrier, preferably by the use of a main pump 15, and a supply manifold 17 for the selective input of such culture carrier requirements as, but not limited to, nutrients 3, buffers 5, fresh medium 7, cytokines 9, growth factors 11, and hormones 13. In this preferred embodiment, the main pump 15 provides fresh fluid carrier to the oxygenator 21 where the fluid carrier is oxygenated and passed through the cell culture chamber 19. The waste in the spent fluid carrier from the cell culture chamber 19 is removed and delivered to the waste 18 and the remaining cell culture carrier is returned to the manifold 17 where it receives a fresh charge, as necessary, before recycling by the pump 15 through the oxygenator 21 to the cell culture chamber 19.

In the culture carrier flow loop 1, the culture carrier is circulated through the living cell culture in the chamber 19 and around the culture carrier flow loop 1, as shown in FIG 1. In this loop 1, adjustments are made in response to chemical sensors (not shown) that maintain constant conditions within the cell culture reactor chamber 19. Controlling carbon dioxide pressures and introducing acids or bases corrects pH. Oxygen, nitrogen, and carbon dioxide are dissolved in a gas exchange system (not shown) in order to support cell respiration. The closed loop 1 adds oxygen and removes carbon dioxide from a circulating gas capacitance. Although FIG. 1 is one preferred embodiment of a culture carrier flow loop that may be used in the present invention, the invention is not intended to be so limited. The input of culture carrier such as, but not limited to, oxygen, nutrients, buffers, fresh medium, cytokines, growth factors, and hormones into a bioreactor can also be performed manually, automatically, or by other control means, as can be the control and removal of waste and carbon dioxide.

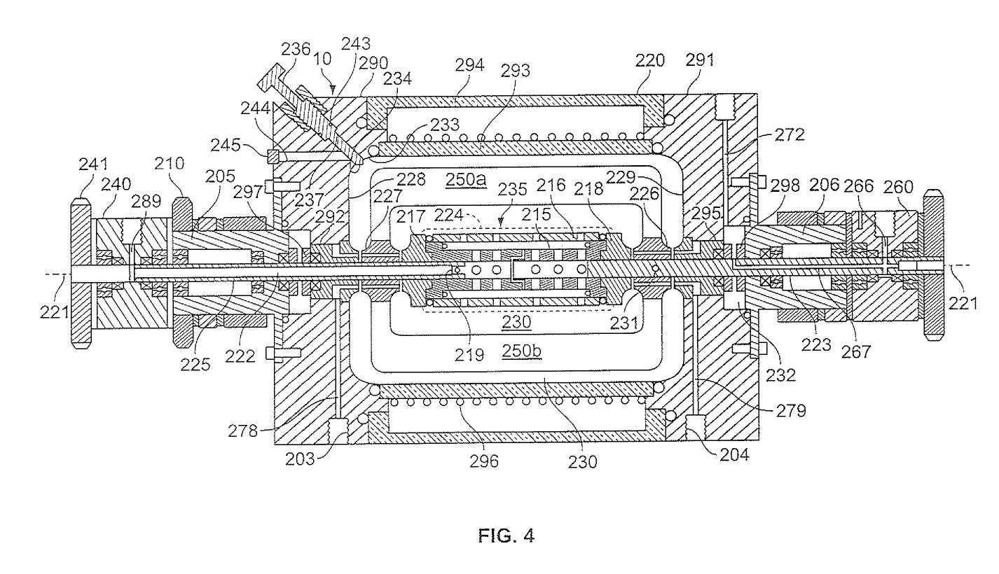

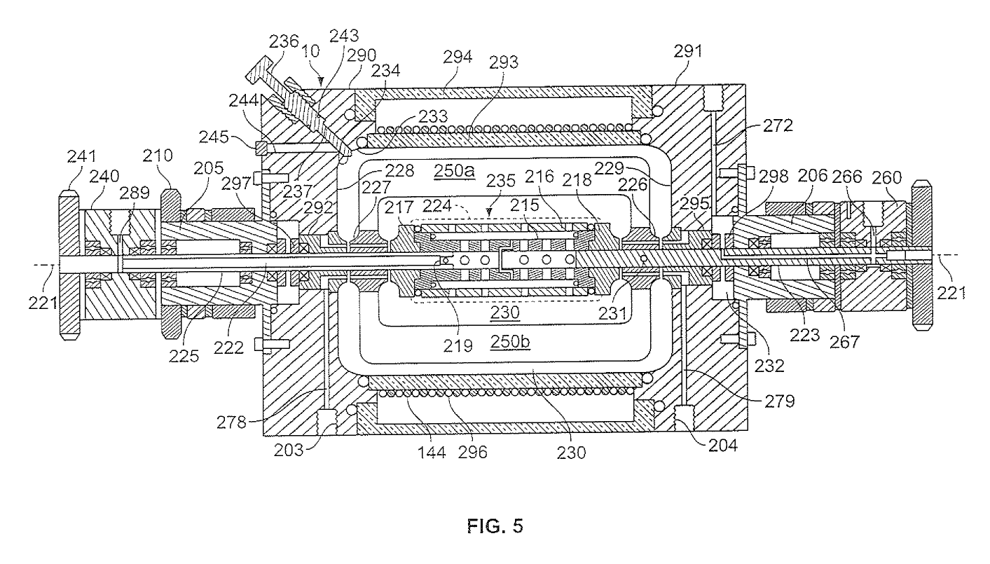

FIGS. 2 and 3 illustrate a preferred embodiment of a TVEMF-bioreactor 10 with an integral time varying electromagnetic force source. FIG. 4 is a cross section of a rotatable TVEMF-bioreactor 10 for use in the present invention in a preferred form. The TVEMF-bioreactor 10 of FIG. 4 is illustrated with an integral time varying electromagnetic force source. FIG. 5 also illustrates a preferred embodiment of a TVEMF-bioreactor with an integral time varying electromagnetic force source. FIGS. 6-8 show a rotating bioreactor with an adjacent time varying electromagnetic force source.

Turning now to FIG. 2, illustrated in FIG. 2 is an elevated side view of a preferred embodiment of a TVEMF-bioreactor 10 of the present invention. FIG. 2 comprises a motor housing 111 supported by a base 112. A motor 113 is attached inside the motor housing 111 and connected by a first wire 114 and a second wire 115 to a control box 116 that has a control means therein whereby the speed of the motor 113 can be incrementally controlled by turning the control knob 117. The motor housing 111 has a motor 113 inside set so that a motor shaft 118 extends through the housing 111 with the motor shaft 118 being longitudinal so that the center of the shaft 118 is parallel to the plane of the earth at the location of a longitudinal chamber 119, preferably made of a transparent material including, but not limited to, plastic.

In this preferred embodiment, the longitudinal chamber 119 is connected to the shaft 118 so that the chamber 119 rotates about its longitudinal axis with the longitudinal axis parallel to the plane of the earth. The chamber 119 is wound with a wire coil 120. The size of the wire coil 120 and number of times it is wound are such that when a square wave current preferably of from 0.1 mA to 1000 mA is supplied to the wire coil 120, a time varying electromagnetic force preferably of from 0.05 gauss to 6 gauss is generated within the chamber 119. The wire coil 120 is connected to a first ring 121 and a second ring 122 at the end of the shaft 118 by wires 123 and 124. These rings 121, 122 are then contacted by a first electromagnetic delivery wire 125 and a second electromagnetic delivery wire 128 in such a manner that the chamber 119 can rotate while the current is constantly supplied to the coil 120. An electromagnetic generating device 126 is connected to the wires 125, 128. The electromagnetic generating device 126 supplies a square wave to the wires 125, 128 and coil 120 by adjusting its output by turning an electromagnetic generating device knob 127.

FIG. 3 is a side perspective view of the TVEMF-bioreactor 10 shown in FIG. 2 that may be used in the present invention.

Turning now to the rotating TVEMF-bioreactor 10 illustrated in FIG. 4 with a culture chamber 230 which is preferably transparent and adapted to contain a peripheral blood mixture therein, further comprising an outer housing 220 which includes a first 290 and second 291 cylindrically shaped transverse end cap member having facing first 228 and second 229 end surfaces arranged to receive an inner cylindrical tubular glass member 293 and an outer tubular glass member 294. Suitable pressure seals are provided. Between the inner 293 and outer 294 tubular members is an annular wire heater 296 which is utilized for obtaining the proper incubation temperatures for cell growth. The wire heater 296 can also be used as a time varying electromagnetic force device to supply a time varying electric field to the culture chamber 230 or, as depicted in FIG. 5, a separate wire coil 144 can be used to supply a time varying electromagnetic force. The first end cap member 290 and second end cap member 291 have inner curved surfaces adjoining the end surfaces 228, 229 for promoting smoother flow of the mixture within the chamber 230. The first end cap member 290, and second end cap member 291 have a first central fluid transfer journal member 292 and second central fluid transfer journal member 295, respectively, that are rotatably received respectively on an input shaft 223 and an output shaft 225. Each transfer journal member 294, 295 has a flange to scat in a recessed counter bore in an end cap member 290, 291 and is attached by a first lock washer and ring 297, and second lock washer and ring 298 against longitudinal motion relative to a shaft 223, 225. Each journal member 294, 295 has an intermediate annular recess that is connected to longitudinally extending, circumferentially arranged passages. Each annular recess in a journal member 292, 295 is coupled by a first radially disposed passage 278 and second radially disposed passage 279 in an end cap member 290 and 291, respectively, to first input coupling 203 and second input coupling 204. Carrier in a radial passage 278 or 279 flows through a first annular recess and the longitudinal passages in a journal member 294 or 295 to permit access carrier through a journal member 292, 295 to each end of the journal 292, 295 where the access is circumferential about a shaft 223, 225.

Attached to the end cap members 290 and 291 are a first tubular bearing housing 205, and second tubular bearing housing 206 containing ball bearings which relatively support the outer housing 220 on the input 223 and output 225 shafts. The first bearing housing 205 has an attached first sprocket gear 210 for providing a rotative drive for the outer housing 220 in a rotative direction about the input 223 and output 225 shafts and the longitudinal axis 221. The first bearing housing 205, and second bearing housing 206 also have provisions for electrical take out of the wire heater 296 and any other sensor.

The inner filter assembly 235 includes inner 215 and outer 216 tubular members having perforations or apertures along their lengths and have a first 217 and second 218 inner filter assembly end cap member with perforations. The inner tubular member 215 is constructed in two pieces with an interlocking centrally located coupling section and each piece attached to an end cap 217 or 218. The outer tubular member 216 is mounted between the first 217 and second inner filter assembly end caps.

The end cap members 217, 218 are respectively rotatably supported on the input shaft 223 and the output shaft 225. The inner member 215 is rotatively attached to the output shaft 225 by a pin and an interfitting groove 219. A polyester cloth 224 with a ten-micron weave is disposed over the outer surface of the outer member 216 and attached to O-rings at either end. Because the inner member 215 is attached by a coupling pin to a slot in the output drive shaft 225, the output drive shaft 225 can rotate the inner member 215. The inner member 215 is coupled by the first 217 and second 218 end caps that support the outer member 216. The output shaft 225 is extended through bearings in a first stationary housing 240 and is coupled to a first sprocket gear 241. As illustrated, the output shaft 225 has a tubular bore 222 that extends from a first port or passageway 289 in the first stationary housing 240 located between seals to the inner member 215 so that a flow of fluid carrier can be exited from the inner member 215 through the stationary housing 240.

Between the first 217 and second 218 end caps for the inner member 235 and the journals 292, 295 in the outer housing 220, are a first 227 and second 226 hub for the blade members 50a and 50b. The second hub 226 on the input shaft 223 is coupled to the input shaft 223 by a pin 231 so that the second hub 226 rotates with the input shaft 223. Each hub 227, 226 has axially extending passageways for the transmittal of carrier through a hub.

The input shaft 223 extends through bearings in the second stationary housing 260 for rotatable support of the input shaft 223. A second longitudinal passageway 267 extends through the input shaft 223 to a location intermediate of retaining washers and rings that are disposed in a second annular recess 232 between the faceplate and the housing 260. A third radial passageway 272 in the second end cap member 291 permits fluid carrier in the recess to exit from the second end cap member 291. While not shown, the third passageway 272 connects through piping and a Y joint to each of the passages 278 and 279.

A sample port is shown in FIG. 4, where a first bore 237 extending along a first axis intersects a corner 233 of the chamber 230 and forms a restricted opening 234. The bore 237 has a counter bore and a threaded ring at one end to threadedly receive a cylindrical valve member 236. The valve member 236 has a complimentarily formed tip to engage the opening 234 and protrude slightly into the interior of the chamber 230. An O-ring 243 on the valve member 236 provides a seal. A second bore 244 along a second axis intersects the first bore 237 at a location between the O-ring 243 and the opening 234. An elastomer or plastic stopper 245 closes the second bore 244 and can be entered with a hypodermic syringe for removing a sample. To remove a sample, the valve member 236 is backed off to access the opening 234 and the bore 244. A syringe can then be used to extract a sample and the opening 234 can be reclosed. No outside contamination reaches the interior of the TVEMF-bioreactor 10.

In operation, carrier is input to the second port or passageway 266 to the shaft passageway and thence to the first radially disposed 278 and second radially disposed passageways 279 via the third radial passageway 272. When the carrier enters the chamber 230 via the longitudinal passages in the journals 292, 294 the carrier impinges on an end surface 228, 229 of the hubs 227, 226 and is dispersed radially as well as axially through the passageways in the hubs 227, 226. Carrier passing through the hubs 227, 226 impinges on the end cap members 217, 218 and is dispersed radially. The flow of entry fluid carrier is thus radially outward away from the longitudinal axis 221 and flows in a toroidal fashion from each end to exit through the polyester cloth 224 and openings in filter assembly 235 to exit via the passageways 266 and 289. By controlling the rotational speed and direction of rotation of the outer housing 220, chamber 230, and inner filter assembly 235 any desired type of carrier action can he obtained. Of major importance, however, is the fact that a clinostat operation can be obtained together with a continuous supply of fresh fluid carrier.

If a time varying electromagnetic force is not applied using the integral annular wire heater 296, it can be applied by another preferred time varying electromagnetic force source. For instance, FIGS. 6-8 illustrate a time varying electromagnetic force device 140 which provides an electromagnetic force to a cell culture in a bioreactor which does not have an integral time varying electromagnetic force, but rather has an adjacent time varying electromagnetic force device. Specifically, FIG. 6 is a preferred embodiment of a time varying electromagnetic force device 140. FIG. 6 is all elevated side perspective of the device 140 which comprises a support base 145, a cylinder coil support 146 supported on the base 145 with a wire coil 147 wrapped around the support 146. FIG. 7 is a front perspective of the time varying electromagnetic force device 140 illustrated in FIG 6. FIG. 8 is a front perspective of the time varying electromagnetic force device 140, which illustrates that in operation, an entire bioreactor 148 is inserted into a cylinder coil support 146 which is supported by a support base 145 and which is wound by a wire coil 147. Since the time varying electromagnetic force device 140 is adjacent to the bioreactor 148, the time varying electromagnetic force device 140 can be reused. In addition, since the time varying electromagnetic force device 140 is adjacent to the bioreactor 148, the device 140 can be used to generate an electromagnetic force in all types of bioreactors, preferably rotating.

In operation, during TVEMF-expansion, a TVEMF-bioreactor 10 of the present invention contains a peripheral blood mixture in the cell culture chamber. During TVEMF-expansion, the speed of the rotation of the peripheral blood mixture-containing chamber may be assessed and adjusted so that the peripheral blood mixture remains substantially at or about the longitudinal axis. Increasing the rotational speed is warranted to prevent wall impact. For instance, an increase in the rotation is preferred if the peripheral blood stem cells in the peripheral blood mixture fall excessively inward and downward on the downward side of the rotation cycle and excessively outward and insufficiently upward on the upward side of the rotation cycle. Optimally, the user is advised to preferably select a rotational rate that fosters minimal wall collision frequency and intensity so as to maintain the peripheral blood stem cell three-dimensional geometry and their cell-to-cell support and cell-to-cell geometry. The preferred speed of the present invention is of from 5 to 120 RPM, and more preferably from 10 to 30 RPM.

The peripheral blood mixture may preferably be visually assessed through the preferably transparent culture chamber and manually adjusted. The assessment and adjustment of the peripheral blood mixture may also be automated by a sensor (for instance, a laser), which monitors the location of the peripheral blood stem cells within a TVEMF-bioreactor 10. A sensor reading indicating too much cell movement will automatically cause a mechanism to adjust the rotational speed accordingly.

Furthermore, in operation the present invention contemplates that an electromagnetic generating device is turned on and adjusted so that the square wave output generates the desired electromagnetic field in the peripheral blood mixture-containing chamber, preferably in a range of from 0.05 gauss to 6 gauss.

Preferably, the square wave has a frequency of about 2 to about 25 cycles/second, more preferably about 5 to about 20 cycles/second, and for example about 10 cycles/second, and the conductor has an RMS value of about 1 to about 1000 mA, preferably about 1 to about 6 mA. However, these parameters are not meant to be limiting to the TVEMF of the present invention, as such may vary based on other aspects of this invention. TVEMF may be measured for instance by standard equipment such as an EN131 Cell Sensor Gauss Meter.

As various changes could be made in rotating bioreactors subjected to a time varying electromagnetic force as are contemplated in the present invention, without departing from the scope of the invention, it is intended that all matter contained herein be interpreted as illustrative and not limiting.

### DETAILED DESCRIPTION OF THE PREFERRED EMBODIMENTS OF THE INVENTION

The following definitions are meant to aid in the description and understanding of the defined terms in the context of the present invention. The definitions are not meant to limit these terms to less than is described throughout this application. Furthermore, several definitions are included relating to TVEMF—all of the definitions in this regard should be considered to complement each other, and not construed against each other.

As used throughout this application, the term “adult stem cell” refers to a pluripotent cell that is undifferentiated and that may give rise to more differentiated cells. With regard to the present invention, an adult stem cell is preferably CD34+/CD38−. Adult stem cells are also known as somatic stem cells, and are not embryonic stem cells directly derived from an embryo.

As used throughout this application, the term “peripheral blood” refers to systemic blood; that is, blood that circulates, or has circulated, systemically in a mammal. The mammal is not a fetus. For the purposes of the present invention, there is no reason to distinguish between blood located at different parts of the same circulatory loop.

As used throughout this application, the term “peripheral blood cell” refers to a cell from peripheral blood. Peripheral blood cells capable of replication may undergo TVEMF-expansion in a TVEMF-bioreactor, and may be present in compositions of the present invention.

As used throughout this application, the term “peripheral blood stem cell” refers to an adult stem cell from peripheral blood. Peripheral blood stem cells are adult stem cells, which as mentioned above are also known as somatic stem cells, and are not embryonic stem cells derived directly from an embryo. Preferably, a peripheral blood stem cell of the present invention is a CD34+/CD38− cell.

As used throughout this application, the term “peripheral blood stem cell composition”, or reference thereto, refers to peripheral blood stem cells of the present invention, either (1) in a number per volume at least 7 times greater than the naturally-occurring peripheral blood source and having the same or very similar three-dimensional geometry and cell-to-cell geometry and cell-to-cell support as naturally-occurring peripheral blood stem cells, and/or (2) having undergone TVEMF-expansion, maintaining the above mentioned geometry and support. With the peripheral blood stem cells is a carrier of some sort, whether a pharmaceutically acceptable carrier, plasma, blood, albumin, cell culture medium, growth factor, copper chelating agent, hormone, buffer, cryopreservative, or some other substance. Reference to naturally-occurring peripheral blood is preferably to compare peripheral blood stem cells of the present invention with their original peripheral blood source. However, if such a comparison is not available, then naturally-occurring peripheral blood may refer to average or typical characteristics of peripheral blood, preferably of the same mammalian species as the source of the peripheral blood stem cells of this invention.

As used throughout this application, the term “peripheral blood mixture” refers to a mixture of peripheral blood cells with a substance that allows the cells to expand, such as a medium for growth of cells that may be placed in a TVEMF-bioreactor (for instance in a cell culture chamber). The peripheral blood cells may be present in the peripheral blood mixture simply by mixing whole peripheral blood with a substance such as a cell culture medium. Also, the peripheral blood mixture may be made with a cellular preparation from peripheral blood, such as a “buffy coat”, as described throughout this application, containing peripheral blood stem cells. Preferably, the peripheral blood mixture comprises CD34+/CD38− peripheral blood stem cells an(i Dulbeeco's medium (DMEM). Preferably, at least half of the peripheral blood mixture is a cell culture medium such as DMEM.

As used throughout this application, the term “TVEMF” refers to “Time Varying Electromagnetic Force”. As discussed above, the TVEMF of this invention is a square wave (following a Fourier curve). Preferably, the square wave has a frequency of about 10cycles/second, and the conductor has an RMS value of about 1 to 1000 mA, preferably 1 to 6 mA. However, these parameters are not meant to be limiting to the TVEMF of the present invention, as such may vary based on other aspects of this invention. TVEMF may be measured for instance by standard equipment such as an EN131 Cell Sensor Gauss Meter.

As used throughout this application, the term “TVEMF-bioreactor” refers to a rotating bioreactor to which TVEMF is applied, as described more fully in the Description of the Drawings, above. The TVEMF applied to a bioreactor is preferably in the range of 0.05 to 6.0 gauss, preferably 0.05-0.5 gauss. See for instance FIGS. 2, 3, 4 and 5 herein for examples (not meant to be limiting) of a TVEMF-bioreactor. In a simple embodiment, a TVEMF-bioreactor of the present invention provides for the rotation of an enclosed peripheral blood mixture at an appropriate gauss level (with TVEMF applied), and allows the pcriplleral blood cells (including stem cells) therein to expand. Preferably, a TVEMF-bioreactor allows for the exchange of growth medium (preferably with additives) and for oxygenation of the peripheral blood mixture. The TVEMF-bioreactor provides a mechanism for growing cells for several days or more. Without being bound by theory, the TVEMF-bioreactor subjects cells in the bioreactor to TVEMF, so that TVEMF is passed through or otherwise exposed to the cells, the cells thus undergoing TVEMF-expansion.

As used throughout this application, the term “TVEMF-expanded peripheral blood cells” refers to peripheral blood cells increased in number per volume after being placed in a TVEMF-bioreactor and subjected to a TVEMF of about 0.05 to 6.0 gauss. The increase in number of cells per volume is the result of cell replication in the TVEMF-bioreactor, so that the total number of cells increase. The increase in number of cells per volume is expressly not due to a simple reduction in volume of fluid, for instance, reducing the volume of blood fiom 70 ml to 10 ml and thereby increasing the number of cells per ml.

As used throughout this application, the term “TVEMF-expanded peripheral blood stem cells” refers to peripheral blood stem cells increased in number per volume after being placed in a TVEMF-bioreactor and subjected to a TVEMF of about 0.05 to 6.0 gauss. The increase in number of stem cells per volume is the result of cell replication in the TVEMF-bioreactor, so that the total number of stem cells in the bioreactor increase. The increase in number of stem cells per volume is expressly not due to a simple reduction in volume of fluid, for instance, reducing the volume of blood from 70 ml to 10 ml and thereby increasing the number of stem cells per ml.

As used throughout this application, the term “TVEMF-expanding” refers to the step of cells in a TVEMF-bioreactor replicating (splitting and growing) in the presence of TVEMF in a TVEMF-(rotating) bioreactor. Peripheral blood stem cells (preferably CD34+/CD38− stem cells) preferably replicate without undergoing further differentiation, so that all or substantially all CD34+/C38− stem cells expanded according to this invention replicate, but do not differentiate, during their time in a bioreactor. “Substantially all” is meant to refer to at least 70%, preferably at least 80%, more preferably at least 90%, even more preferably at least 95%, even more preferably at least 97%, and most preferably at least 99% of CD34+CD38− cells do not differentiate such that they are no longer CD34+/CD38− during TVEMF-expansion.

As used throughout this application, the term “TVEMF-expansion” refers to the process of increasing the number of peripheral blood cells in a TVEMF-bioreactor, preferably peripheral blood stem cells, by subjecting the cells to a TVEMF of about 0.05 to about 6.0 gauss. Preferably, the increase in number of peripheral blood stem cells, is at least 7 times the number per volume of the original peripheral blood source. The expansion of peripheral blood stem cells in a TVEMF-bioreactor according to the present invention provides for peripheral blood stem cells that maintain, or have the same or essentially the same, three-dimensional geometry and cell-to-cell support and cell-to-cell geometry as peripheral blood stem cells prior to TVEMF-expansion. Other aspects of TVEMF-expansion may also provide the exceptional characteristics of the peripheral blood stem cells of the present invention. Not to be bound by theory, TVEMF-expansion not only provides for high concentrations of peripheral blood stem cells that maintain their three-dimenisional geometry and cell-to-cell support. Not to be bound by theory, TVEMF may affect some properties of stem cells during TVEMF-expansion, for instance up-regulation of genes promoting growth, or down regulation of genes preventing growth. Overall, TVEMF-expansion results in promoting peripheral blood stem cell growth but not differentiation.

As used throughout this application, the term “TVEMF-expanded cell” refers to a cell that has been subjected to the process of TVEMF-expansion.

Throughout this application, reference to the repair of tissue, treatment of disease or condition, are not meant to be exclusive but rather relate to the objective of overall tissue repair where improvement in tissue results from administration of stem cells as discussed herein. While the present invention is directed in part to treatment of diseases or conditions that are symptomatic, and possibly life-threatening, the present inventi on is also meant to inelude treatment of minor repair, and even prevention/prophylaxis of a disease or condition by early introduction of expanded stem cells, before symptoms or problems in the mammal's (preferably human's) health are noticed.

Throughout this application, the terms “repair”, “replenish” and “regenerate” are used. These terms are not meant to be mutually exclusive, but rather related to overall tissue repair.

As used throughout this application, the term “toxic substance” or related terms may refer to substances that are toxic to a cell, preferably a peripheral blood stem cell; or toxic to a patient. In particular, the term toxic substance refers to dead cells, macrophages, as well as substances that may be unique or unusual in peripheral blood (for instance, sickle cells or other t issue or waste). Other toxic substances are discussed throughout this application. Removal of toxic substances from blood is well-known in the art, in particular art relating to the introduction of blood products to a patient.

As used throughout this application, the term “apheresis of bone marrow” refers to inserting a needle into bone and extracting bone marrow. Such apheresis is well-known in the art.

As used throughout this application, the term “autologous” refers to a situation in which the dionor (source of peripheral blood stem cells prior to expansion) and recipient are the same mammal.

As used throughout this application, the term “allogeneic” refers to a situation in which the donor (source of peripheral blood stein cells prior to expansion) and recipient are not the same mammal.

As used throughout this application, the term “CD34+” refers to the presence of a surface antigen (CD34) on the surface of a blood cell. CD34 protein is present on the surface of hematopoicetic stem cells in all states of development.

As used throughout this application, the term “CD38−” refers to the lack of a surface antigen (CD38) on the surface of a blood cell, CD38 is not present on the surface of stem cells of the present invention.

As used throughout this application, the term “cell-to-cell geometry” refers to the geometry of cells including the spacing, distance between, and physical relationship of the cells relative to one another. For instance, TVEMF-expanded stem cells of this invention stay in relation to each other as in the body. The expanded cells are within the bounds of natural spacing between cells, in contrast to for instance two-dimensional expansion containers, where such spacing is not kept.

As used throughout this application, the term “cell-to-cell support” refers to the support one cell provides to an adjacent cell. For instance, healthy tissue and cells maintain interactions such as chemical, hormonal, neural (where applicable/appropriate) with other cells in the body. In the present invention, these interactions are maintained within normal functioning parameters, meaning they do not for instance begin to send toxic or damaging signals to other cells (unless such would be done in the natural blood environment).

As used throughout this applpication, the term “three-dimensional geometry” refers to tle geometry of cells in a three-dimensional state (same as or very similar to their natural state), as opposed to two-dimensional geometry for instance as found in cells grown in a Petri dish, where the cells become flattened and/or stretched.

For each of the above three definitions, relating to maintenance of cell-to-cell support and geometry and three dimensional geometry of stem cells of the present invention, the term “essentially the same” means that normal geometry and support are provided in TVEMF-expanded cells of this invention, so that the cells are not changed in such a way as to be for instance disfunctional, unable to repair tissue, or toxic or harmful to other cells.

The present invention is directed to providing a rapidly available source of TVEMF-expanded peripheral blood stem cells for repairing, replenishing and regenerating tissue in mammals, preferably humans. This invention may be more fully described by the preferred embodiment(s) as hereinafter described, but is not intended to be limited thereto.

Operative Method—Preparing a TVEMF-Expanded Peripheral Blood Stem Cell Composition, and Using the Composition

In a preferred embodiment of this invention, a method is described for preparing TVEMF-expanded peripheral blood stem cells that can assist the body in repairing, replacing and regenerating tissue or be useful in research or treatment of disease.

Peripheral blood is to be collected from a mammal, preferably a primate mammal, and more preferably a human, for instance as described throughout this application and as known in the art, and preferably via a syringe as well known in the art. Peripheral blood may be collected, for instance, and expanded immediately or cryopreserved for later use. Peripheral blood would only be removed from a human in an amount that would not be threatening to the subject. Preferably, about 10 to about 500 ml peripheral blood is collected; more preferably, 100-300 ml, even more preferably, 150-200 ml. The collection of peripheral blood according to this invention is not meant to be limiting, but can also include for instance other means of directly collecting mammalian peripheral blood, pooling peripheral blood from one or more sources, indirectly collecting peripheral blood for instance by acquiring the blood from a commercial or other source, including for instance cryopreserved blood from a “blood bank”.

Typically, when directly collected from a mammal, peripheral blood is drawn into one or more syringes, preferably containing anticoagulants. The blood may be stored in the syringe or transferred to another vessel. Peripheral blood may then be separated into its parts; white blood cells, red blood cells, and plasma. This is either done in a centrifuge (an apparatus that spins the container of blood until the blood is divided) or by sedimentation (the process of injecting sediment into the container of blood causing the blood to separate). Second, once the peripheral blood is divided with the red blood cells (RBC) on the bottom, white blood cells (WBC) in the middle, and the plasma on top, the white blood cells are removed for storage. The middle layer, also known as the “buffy coat” contains the peripheral blood stem cells of interest; the other parts of the blood are not needed. For some blood banks, this will be the extent of their processing. However, other banks will go on to process the buffy coat by removing the mononuclear eells (in this case, a subset of white blood cells) from the WBC. While not everyone agrees with this method, there is less to store and less cryogenic nitrogen is needed to store the cells.

Another method for separating peripheral blood cells is to subject all of the collected peripheral blood to one or more (preferably three) rounds of continuous flow leukapheresis in a separator such as a Cobe Spectra cell separator. Such processing will separate peripheral blood cells having one nucleus from other peripheral blood cells. The stem cells are part of the group having one nucleus. Other methods for the separation of blood cells are known in the art.

It is preferable to remove the RBC from the peripheral blood sample. While people may have the same HLA type (which is needed for the transplanting of stem cells), they may not have the same blood type. By removing the RBC, adverse reactions to a stem cell transplant can be minimized. By eliminating the RBC, therefore, the stem cell sample has a better chance of being compatible with more people. RBC can also burst when they are thawed, releasing free hemoglobin. This type of hemoglobin can seriously affect the kidneys of people receiving a transplant. Additionally, the viability of the stem cells are reduced when RBC rupture.

Also, particularly if storing peripheral blood cryogenically or transferring the blood to another mammal, the blood may be tested to ensure no infectious or genetic diseases, such as HIV/AIDS, hepatitis, leukemia or immune disorder, is present. If such a disease exists, the blood may be discarded or used with associated risks noted for a future user to consider.

In still another embodiment of this invention, blood cells may be obtained from a donor. Prior to collection, the donor is preferably treated with G-CSF (preferably in an amount of 0.3 ng to 5 ug, more preferably 1 ng/kg to 100 ng/kg, even more preferably 5 ng/kg to 20 ng/kg, and even more preferably 6 ng/kg) every 12 hr over 3 days and then once on day 4. In a preferred method, a like amount of GM-CSF is also administered. Other alternatives are to use GM-CSF alone, or other growth factor molecules, interleukins. Blood is then collected from the donor, and may be used whole in a peripheral blood mixture or first separated into cellular parts as discussed throughout this application, where the cellular part including stem cells (CD34+/CD38−) is used to prepare the peripheral blood mixture to be expanded. Cells may be separated, for instance, by subjecting the donor's total blood volume to 3 rounds of conitinuous-flow leukapheresis through a separator, such as a Cobe Spectra cell separator. Preferably, the expanded stem cells are reintroduced into the same donor, where the donor is in need of tissue repair as discussed herein. However, allogeneic introduction may also be used, as also indicated herein. Other pre-collection administrations will also be evident to those skilled in the art.

Preferably, red blood cells are removed from the peripheral blood and the remaining cells including peripheral blood stem cells are placed with an approlpriate media in a TVEMF-bioreactor (see “peripheral blood mixture”) such as that described herein. In a more preferred embodiment of this invention, only the “buffy coat” (which includes peripheral blood stem cells, as discussed throughout this application) described above is the cellular material placed in the TVEMF-bioreactor. Other embodiments include removing other non-stem cells and components of the peripheral blood, to prepare different peripheral blood cell preparation(s). Such a peripheral blood cell preparation may even have, as the only remaining peripheral blood component, CD34+/CD38− peripheral blood stem cells. IRemoval of non-stem cell types of peripheral blood cells may be achieved through negative separation techniques, such as but not limited to sedimentation and centrifugation. Many negative separation methods are well-known in the art. However, positive selection techniques may also be used, and are preferred in this invention. Methods for removing various components of the blood and positively selecting for CD34+/CD38− are known in the art, and may be used so long as they do not lyse or otherwise irreversibly harm the desired peripheral blood stem cells. For instance, an affinity method selective for CD34+/CD38− may be used. Preferably, a “buffy coat” as described above is prepared from peripheral blood, and the CD34+/CD38− cells therein separated from the buffy coat for TVEMF-expansion.

The collected peripheral blood, or desired cellular parts as discussed above, must be placed into a TVEMF-bioreactor for TVEMF-expansion to occur. As discussed above, the term “peripheral blood mixture” comprises a mixture of peripheral blood (or desired cellular part, for instance peripheral blood without red blood cells, or “buffy coat” cells, or preferably CD34+/CD38− peripheral blood stem cells isolated from peripheral blood) with a substance that allows the cells to expand, such as a m-edium for growth of cells, that will be placed in a TVEMF-bioreactor. Cell culture media, media that allow cells to grow and expand, are well-known in the art. Preferably, the substance that allows the cells to expand is cell culture media, more preferably Dulbecco's medium. The components of the cell media must, of course, not kill or damage the stem cells. Other componenets may also be added to the peripheral blood mixture prior to or during TVEMF-expansion. For instance, the peripheral blood may be placed in the bioreactor with Dulbccco's medium and further supplemented with 5% (or some other desired amount, for instance in the range of about 1% to about 10%) of human serum albumin. Other additives to the peripheral blood mixture, including but not limited to growth factor, copper chelating agent, cytokine, hormone and other substances that may enhance TVEMF-expansion may also be added to the peripheral blood outside or inside the bioreactor before being placed in the bioreactor. Preferably, the entire volume of a peripheral blood collection from one individual (preferably human peripheral blood in an amount of about 1.0 ml to about 500 ml, more preferably about 100 ml to about 300 ml, even more preferably about 150 to about 200 ml peripheral blood) is mixed with a cell culture medium such as Dulbecco's medium (DMEM) and supplemented with 5% human serum albumin to prepare a peripheral blood mixture for rVEMF-expansioni. For instance, for a 50 to 100 ml peripheral blood sample, preferably about 25 to about 100 ml DMEM/5% human serum albumin is used, so that the total volume of the peripheral blood mixture is about 75 to about 200 ml when placed in the bioreactor. As a general rule, the more peripheral blood that may be, collected, the better; for instance, if a collection from one individual results in more than 200 ml, the use of all of the stem cells in that peripheral blood is preferred. Where a larger volume is available, for instance by pooling peripheral blood (from the same or different source), more than one dose may be preferred. The use of a perfusion TVEMF-bioreactor is particularly useful when peripheral blood collections are pooled and TVEMF-expanded together.

A copper chelating agent of the present invention may be any non-toxic copper chelating agent, and is preferably Penicillamine or Trientine Hydrochloride. More preferably, the Penicillamine is D(−)-2-Amino-3-Mercaptor-3-Methylbutanic Acid (Sigma-Aldrich), dissolved in DMSO and added to the peripheral blood mixture in an amnount of about 10 ppm. The copper chelating agent may also be administered to a mammal, where peripheral blood will then be directly collected from the mammal, Preferably such administration is more than one clay, more preferably more than two days, before collecting peripheral blood from the mammal. The purpose of the copper chelating agent, whether added to the peripheral blood mixture itself or administered to a blood donor mammal, or both, is to reduce the amount of copper in the peripheral blood prior to TVEMF-expansion. Not to be bound by theory, it is believed that the decrease in amount of available copper may enhance TVFMF-expansion.

The term “placed into a TVEMF-bioreactor” is not meant to be limiting—the peripheral blood mixture may be made entirely outside of the bioreactor and then the mixture placed inside the bioreactor. Also, the peripheral blood mixture may be entirely mixed inside the bioreactor. For instance, the peripheral blood (or a cellular portion thereof) may be placed in the bioreactor and supplemented with Dulbecco's medium and 5% human serum albumin either already in the bioreactor, added simultaneously to the bioreactor, or added after the peripheral blood to the bioreactor.

A preferred peripheral blood mixture of the present invention to be placed in a TVEMF-bioreactor comprises the following: CD34+/CD38− stem cells isolated from the buffy coat of a peripheral blood sample; and Dulbecco's meldium which, with the CD34+/CD38− cells, is about 150-250 ml, preferably about 200 ml total volume. Even more preferably, G-CSF (Granulocyte-Colony Stimulating Factor) is included in the peripheral blood mixture. Preferably, G-CSF is present in an amount sufficient to enhance TVEMF-expansion of peripheral blood stem cells. Even more preferably, the amount of G-CSF present in the peripheral blood mixture prior to TVEMF-expansion is about 25 to about 200 ng/ml peripheral blood mixture, more preferably about 50 to about 150 ng/ml, and even more preferably about 100 ng/ml .

The TVEMF-bioreactor vessel (containing the peripheral blood mixture including the peripheral blood stem cells) is rotated at a speed that provides for suspension of the peripheral blood stem cells to maintain their three-dimensional geometry and their cell-to-cell support antd cell-to-cell geometry. Preferably, the rotational speed is 5-120 rpm; more preferably, from 10-30 rpm. These rotational speeds are not intended to be limiting; rotational speed will depend at least in part on the type of bioreactor and size of cell culture chamber and sample placed therein, During the time that the cells are in the TVEMF-bioreactor, they are preferably fed nutrients and fresh media (for instance, DMEM and 5% human serum albumin; see above discussions of fluid carriers), exposed to hormones, cytokines, and/or growth factors (preferably G-CSF); and toxic materials are removed. The toxic materials removed from peripheral blood cells in a TVEMF-bioreactor include toxic granular material of dying cells and toxic material of gratiulocytes and macrophiages. The TVEMF-expansion of the cells is controlled so that the cells preferably expand (increase in number per volume) at least seven times. Preferably, peripheral blood stem cells (with other cells, if present) undergo TVEMF-expansion for at least 4 days, preferably about 7 to about 14 days, more preferably about 7 to about 10 days, even more preferably about 7 days. TVEMF-expansion may continue in a TVEMF-bioreactor for up to 160 days. While TVEMF-expansion may occur for even longer than 160 days, such a lengthy expansion is not a preferred embodiment of the present invention,

Preferably, TVEMF-expansion is carried out in a TVEMF-bioreactor at a temperature of about 26° C. to about 41° C. and more preferably, at a temperature of about 37° C.

One method of monitoring the overall expansion of cells undergoing TVEMF-expansion is by visual inspection. Peripheral blood stem cells are typically dark red in color. Preferably, the medium used to form the peripheral blood mixture is light or clear in color. Once tme bioreactor begins to rotate and the TVEMF is applied, the cells preferably cluster in the center of the bioreactor vessel, with the medium surrounding the colored cluster of cells. Oxygenation and other nutrient additions often do not cloud the ability to visualize the cell cluster through a visualization (typically clear plastic) window built into the bioreactor. Formation of the cluster is important for helping the stem cells maintain their three-demensional geometry and cell-to-cell support and cell-to-cell geometry; if the cluster appears to scatter and cells begin to contact the wall of the bioreactor vessel, the rotational speed is increased (manually or automatically) so that the centralized cluster of cells may form again. A measurement of the visualizable diameter of the cell cluster taken soon after formation may be compared with later cluster diameters, to indicate the approximate number increase in cells in the TVEMF-bioraector. Measurement of the increase in the number of cells during TVEMF expansion may also be taken in a number of ways, as known in the art for conventional bioreactors. An automatic sensor could also be included in the TVEMF-bioreactor to monitor and measure the increase in cluster size.

The TVEMF-expansion process may be carefully monitored, for instance by a laboratory expert, who may check cell cluster formation to ensure the cells remain clustered inside the bioreactor and will increase the rotation of the bioreactor when the cell cluster begins to scatter. An automatic system for monitoring the cell cluster and viscosity of the peripheral blood mixture inside the bioreactor may also monitor the cell clusters. A change in the viscosity of the cell cluster may become apparent as early as 2 days after beginning the TVEMF-expansion process, and the rotational speed of the TVEMF-bioreactor may be increased around that time. The TVEMF-bioreactor speed may vary throughout TVEMF-expansion. Preferably, the rotational speed is tim ely adctusted so that the cells undergoing TVEMF-expansion do not contact the sides of the TVEMF-bioreactor vessel.

Also, a laboratory expert may, for instance once a day, during TVEMF-expansion, or once every two days, manually (for instance with a syringe) insert fresh media and preferably other desired additives such as nutrients andl growth factors, as discussed above, into the bioreactor, and draw off the old media containing cell wastes and toxins. Also, fresh media and other additives may be automatically pumped into the TVEMF-bioreactor during TVEMF-expanson, and waste automatically removed.

Peripheral blood stem cells may increase to at least seven times their original number about 7 to about 14 days after being placed in the TVEMF-bioreactor and TVEMF-expanded. Preferably, the TVEMF-expansion occurs for about 7 to 10 days, and more preferably about 7 days. Measurement of the number of stem cells does not need to be taken during TVEMF-expansion therefore. As indicated above and throughout this application, TVEMF-expanded peripheral blood stem cells of the present invention have the same or essentially the same three-dimensionial geometry and cell-to-cell support and cell-to-cell geometry as naturally-occurring, non-expanded peripheral blood stem cells.

Upon completion of TVEMF-expansion, the cellular material in the TVEMF-bioreactor comprises the stem cells of the present invention, in a composition of the present invention. Various substances may be removed from or added to the composition for further use. Another embodiment of the present invention relates to an ex vivo mammalian peripheral blood stem cell composition that functions to assist a body system or tissue to repair, replenish and regenerate tissue, for example, the tissues described throughout this application. The composition comprises TVEMF-expanded peripheral blood stem cells, preferably in an amount of at least seven times the number per volume of peripheral blood stem cells per volume as in the peripheral blood from which it originated. For instance, preferably, if a number× of peripheral blood stem cells was placed in a certain volume into a TVEMF-bioreactor, then after TVEMF-expansion, the number of peripheral blood stein cells in the TVEMF-bioreactor will be at least 7×(barring removal of cells during the expansion process). While this at-least-seven-times-expansion is not necessary for this invention to work, this expansion is particularly preferred for therapeutic purposes. For instance, the TVEMF-expanded cells may be only in amount of 2 times the number of peripheral blood stem cells in the naturally-occurring peripheral blood, if desired. Preferably, TVEMF-expanded cells are in a range of about 4 times to about 25 times the number per volume of peripheral blood stem cells in naturally-occurring peripheral blood. The present invention is also directed to a composition comprising peripheral blood stem cells from a mammal, wherein said peripheral blood stem cells are present in a number per volume that is at least 7 times greater than naturally-occurring peripheral blood from the mammal; and wherein the peripheral blood stem cells have a three-dimensional geometry and cell-to-cell support and cell-to-cell geometry that is the same or similar to or essentially the same as stem cells of the naturally-occurring peripheral blood. A composition of the present invention may include a pharmaceutically acceptable carrier; including but not limited to plasma, blood, albumin, cell culture medium, growth factor, copper chelating agent, hormone, buffer or cryopreservative. “Pharmaceutically acceptable carrier” means an agent that will allow the introduction of the stem cells into a mammal, preferably a human. Such carrier may include substances mentioned herein, including in particular any substances that may be used for blood transfusion, for instance blood, plasma, albumin; also, saline or buffer (preferably buffer supplemented with albumin), preferably from the mammal to which the composition will be introduced. The term “introduction” of a composition to a mammal is meant to refer to “administration” of a composition to an animal. Preferably, administration of stem cells of the present invention to a mamnmal is performed by intravenous injection. However, other forms of administration may be used, including for instance injection directly into an organ or near a site needing repair, rectal administration (particularly for a colonic disorder), and other methods for instance such as those well-known in the art, preferably to introduce stem cells to an immediate area in need of repair. Even more preferably, injection occurs with an acceptable amount G-CSF, for instance in an amount of 0.3 ng to 5 ug, more preferably 1 ng/kg to 100 ng/kg, even more preferably 5 ng/kg to 20 ng/kg, and even more preferably 6 ng/kg. Administration of stem cells in a composition of the present invention may occur with pharmaceutically carriers as described in the general state of the art. The amount of stem cells expanded according to the present invention to be administered in a composition is a therapeutically effective amount (also discussed below) of preferably at least 1000 stem cells, more preferably at least 10⁴ stem cells, even more preferably at least 10⁵ stem cells, and even more preferably in an amount of at least 10⁷ to 10⁹ stem cells, or even more stem cells such as 10¹² stem cells. Administration of such numbers of expanded stem cells may be in one or more doses. As indicated throughout this application, the number of stem cells administered to a patient may be limited to the number of stem cells originally available in source blood, as multiplied by expansion according to this invention. Without being bound by theory, it is believed that stem cells not used by the body after administration will simply be removed by natural body systems. “Acceptable carrier” generally refers to any substance the peripheral blood stem cells of the present invention may survive in, i.e. that is not toxic to the cells, whether after TVEMF-expansion, prior to or after cryopreservation, prior to introduction (administration) into a mammal. Such carriers are well known in the art, and may include a wide variety of substances, including substances described for such a purpose throughout this application. For instance, plasma, blood, albumin, cell culture medium, buffer and cryopreservative are all acceptable carriers of this invention. The desired carrier may depend in part on the desired use

Other expansion methods known in the art (none of which use TVEMF) do not provide an expansion of peripheral blood stein cells in the amount of at least 7 times that of naturally-occurring peripheral blood while still maintaining the peripheral blood stem cells three-dimensional geometry and cell-to-cell support. TVEMF-expanded peripheral blood stem cells have the same or essentially the same, or maintain, the three-dimensional geometry and the cell-to-cell support and cell-to-cell geometry as the peripheral blood from which they originated. The composition may comprise TVEMF-expanded peripheral blood stem cells, preferably suspended in Dulbecco's medium or in a solution ready for cryopreservation. The composition is preferably free of toxic granular material, for example, dying cells and the toxic material or content of granulocytes and macrophages. The composition may be a cryopreserved composition comprising TVEMF-expanded peripheral blood stem cells by decreasing the temperature of the composition to a temperature of from −120° C. to −196° C. and maintaining the cryopreserved composition at that temperature range until needed for therapeutic or other use. As discussed below, preferably, as much toxic material as is possible is removed from the composition prior to cryopreservation.

Another embodiment of the present invention relates to a method of regenerating tissue and/or treating diiseases such as auto-immune diseases (as discussed above) with a composition of TVEMF-expanded peripheral blood stem cells, either having undergone cryopreservation or soon after TVEMF-expansion is complete. The cells may be introduced into a mammalian body, preferably human, for instance injected intravenously or directly into the tissue to be repaired, allowing the body's natural system to repair and regenerate the tissue. Preferably, the composition to be introduced into the mammalian body is free of toxic material and other materials that may cause an adverse reaction to the administered TVEMF-expanded peripheral blood stem cells. The method (and composition) can potentially be used to repair a mammalian, preferably human, vital organ and other tissue, with such potential use including but not limited to liver tissue, heart tissue, hematopoictic tissue, blood vessels, skin tissue, mnuscle tissue, gut tissue, pancreatic tissue, central nervous system cells, bone, cartilage tissue, connective tissue, pulmonary tissue, spleen tissue, brain tissue and other body tissue. The cells are readily available for treatment or research where such treatment or research requires the individual's blood cells, especially if a disease has occurred and cells free of the disease are needed.

### EXAMPLE I

### Actual TVEMF-Expansion of Cells in a TVEMF Bioreactor

Peripheral blood was collected and peripheral blood cells expanded as shown in Table 1, below.

A) Collection and Maintenance of Cells

Human peripheral blood (75 ml; about 0.75×10⁶ cells/ml) was obtained from human donors by syringe as described above and suspended in about 75 ml Iscove's modified Dulbecco's medium (IMDM) (GIBCO, Grand Island, N.Y.) supplemented with 20% of 5% human albumin (HA), 100 ng/ml recombinant human G-CSF (Amgen Inc., Thousand Oaks, Calif.), and 100 ng/ml recombinant human stem cell factor (SCF) (Amgen). The peripheral blood mixture was placed in a TVEMF-bioreactor as shown in FIGS. 2 and 3 herein. TVEMF-expansion occurred at 37° C., 6% CO₂, with a normal air O₂/N ratio. The TVEMF-bioreactor was rotated at a speed of 10 rotations per minute (rpm) initially, then adjusted as needed, as described throughout this application, to keep the peripheral blood cells suspended in the bioreactor. A time varying current of 6 mA was applied to the bioreactor. The square wave TVEMF applied to the peripheral blood mixture was about 0.5 Gauss. (frequency: about 10 cycles/sec).

Culture media in the peripheral blood mixture in the TVEMF-bioreactor was changed/freshened every one to two days. At day 10, the cells were removed from the TVEMF-bioreactor and washed with PBS and analyzed. The results are as set forth in Table 1. Control data refers to a sample of human peripheral blood that has not been expanded; Expanded Sample refers to the respective control sample after TVEMF-expansion.

TABLE 1

| | | |
|---|---|---|
| Control 1 | Cell Count 310,000 | Viability 99% |
| Control 2 | Cell Count 305,000 | Viability 98% |
| Control 3 | Cell Count 325,000 | Viability 100% |
| Control 4 | Cell Count 340,000 | Viability 98% |
| Control 5 | Cell Count 325,000 | Viability 98% |
| Control 6 | Cell Count 330,000 | Viability 98% |
| Control 7 | Cell Count 315,000 | Viability 99% |
| Control 8 | Cell Count 350,000 | Viability 98% |
| Control 9 | Cell Count 320,000 | Viability 98% |
| Control 10 | Cell Count 300,000 | Viability 98% |
| Expanded Sample 1 | Cell Count 3,200,000 | Viability 98% |
| | Corresponding CD34+ | |
| | increase: yes | |
| Expanded Sample 2 | Cell Count 3,400,000 | Viability 100% |
| | Corresponding CD34+ | |
| | increase: yes | |
| Expanded Sample 3 | Cell Count 3,550,000 | Viability 100% |
| | Corresponding CD34+ | |
| | increase: yes | |
| Expanded Sample 4 | Cell Count 3,500,000 | Viability 98% |
| | Corresponding CD34+ | |
| | increase: yes | |
| Expanded Sample 5 | Cell Count 3,450,000 | Viability 99% |
| | Corresponding CD34+ | |
| | increase: yes | |
| Expanded Sample 6 | Cell Count 3,400,000 | Viability 98% |
| | Corresponding CD34+ | |
| | increase: yes | |
| Expanded Sample 7 | Cell Count 3,200,000 | Viability 98% |
| | Corresponding CD34+ | |
| | increase: yes | |
| Expanded Sample 8 | Cell Count 3,550,000 | Viability 99% |
| | Corresponding CD34+ | |
| | increase: yes | |
| Expanded Sample 9 | Cell Count 3,400,000 | Viability 99% |
| | Corresponding CD34+ | |
| | increase: yes | |
| Expanded Sample 10 | Cell Count 3,500,000 | Viability 98% |
| | Corresponding CD34+ | |
| | increase: yes | |

As may be seen from Table 1, TVEMF-expansion of peripheral blood cells resulted in roughly a 10-fold increase in the number of cells over 10 days, as compared to non-expanded control. Fhe culture media where the cells were growing was changed/freshesned once every 1-2 days.

B) Analysis of TVEMF-Expanded Cells

Total cell counts of Control and Expanded Samples were obtained with a counting chamber (a device such as a hemocytometer used by placing a volume of either the control cell suspension or expanded sample on a specially-made microscope slide with a microgrid and counting the number of cells in the sample). The results of the total cell counts in Control samples and in Expanded Samples after 10 days of TVEMF-expansion are shown in Table 1.

The indication of corresponding CD34+ increase in Table 1 was determined as follows: CD34+ cells of the Expanded Samples were separated from other cells therein with a Human CD34 Selection Kit (EasySep positive selection, StemCell Technologies), and counted with a counting chamber as indicated above and confirmed with FACScan flow cytometer (Becton-Dickinson). CFU-GEMM and CFU-GM were counted by clonogenic assay. Cell viability (where a viable cell is alive and a non-viable cell is dead) was determined by trypan blue exclusion test. The answer of “yes” in all Expanded Samples indicates that the number of CD34+ cells increased in amounts corresponding to the total cell count.

C) Increase in Amount of Hematopoietic Colony-Forming Cells

Incubation of the donors' peripheral blood cells in this TVEMF-expansion tissue culture systemn significantly increases the numbers of hematopoietic colony-forming cells. As determined in a separate assay, a constant increase in the numbers of CFU-GM (up to 7-fold) and CFU-GEMM (up to 9-fold) colony-forming cells is observed up to day 7 with no clear plateau.

D) Increase in CD34+ Cells

Incubation of MNCs from normal donors in this TVEMF-expansion tissue culture system significantly increases the numbers of CD34+ cells. As determined in a separate assay, the average number of CD34+ cells increased 10-fold by day 6 of culture and plateaus on that same day.

### Operative Method—Cryopreservation

As mentioned above, peripheral blood is to be collected from a mammal, preferably a human. Red blood cells, at least, are preferably removed from the peripheral blood. The peripheral blood stem cells (with other cells and media as desired) are placed in a TVEMF-bioreactor, subjected to a time varying electromagnetic force and expanded. If RBCs were not removed prior to TVEMF-expansion, preferably they are removed after TVEMF-expansion. The TVEMF-expanded cells may be cryogenically preserved. Further details relating to a method for the cryopresevation of TVEMF-expanded peripheral blood stem cells, and compositions comprising such cells are provided herein and in particular below.

After TVEMF-expansion, the TVEMF-expanded cells, including TVEMF-expanded peripheral blood stem cells, may be transferred into at least one cryopreservation container containing at least one cryoprotective agent. The TVEMF-expanded peripheral blood stem cells are preferably first washed with a solution (for instance, a buffer solution or the desired cryopreservative solution) to remove media and other components present during TVEMF-expansion, and then preferably mixed in a solution that allows for cryopreservation of the cells. Such solution is commonly referred to as a cryopreservative, cryoprservation solution or cryoprotectant. The cells are transferred to an appropriate cryogenic container and the container decreased in temperature to generally from −20° C. to −196° C., preferably about −130° C. to about −150° C., and maintained at that temperature. Preferably, this decrease in temperature is done slowly and carefully, so as to not damage, or at least to minimize damage, to the stem cells during the freezing process. When needed, the temperature of the cells (about the temperature of the cryogenic container) is raised to a temperature compatible with introduction of the cells into the human body (generally from around room temperature to around body temperature), and the TVEMF-expanded cells may be introduced into a mammalian body, preferably human, for instance as discussed throughout this application.

Freezing cells is ordinarily destructive. Not to be bound by theory, onl cooling, water within the cell freezes. Injury then may occur by osmotic effects on the cell membrane, cell dehydration, solute concentration, and ice crystal formation. As ice forms outside the cell, available water is removed from solution and withdrawn from the cell, causing osmotic dehydration and raised solute concentration that may eventually destroy the cell. (For a discussion, see Mazur, P., 1977, Cryobiology 14:251-272.)

Different materials have different freezing points. Preferably, a peripheral blood stem cell composition ready for cryopreservation contains as few contaminating substances as possible, to minimize cell wall damage from the crystallizaton and freezing process.

These injurious effects can be reduced or even circumvented by (a) use of a cryoprotective agent, (b) control of the freezing rate, and (c) storage at a temperature sufficiently low to minimize degradative reactions.

The inclusion of cryopreservation agents is preferred in the present invention. Cryoprotective agents which can be used include but are not limited to a sufficient amount of dimethyl sulfoxide (DMSO) (Lovelock, J. E. and Bishop, M. W. H., 1959, Nature 183:1394-1395; Ashwood-Smith, M. J., 1961, Nature 190:1204-1205), glycerol, polyvinylpyrrolidine (Rinfret, A. P., 1960, Ann. N.Y. Acad. Sci. 85:576), polyethylene glycol (Sloviter, H. A. and Ravdin, R. G., 1962, Nature 196:548), albumin, dextran, sucrose, ethylene glycol, i-erythritol, D-ribitol, D-mannitol (Rowe, A. W., et al., 1962, Fed. Proc. 21:157), D-sorbitol, i-inositol, D-lactose, choline chloride (Bender, M. A., et al., 1960, J. Appl. Physiol. 15:520), amino acid-glucose solutions or amino acids (Phan The Tran and Bender, M. A., 1960, Exp. Cell Res. 20:651), methanol, acetamide, glycerol monoacetate (Lovelock, J. E., 1954, Biochem J. 56:265), and inorganic salts (Phan The Tran and Bender, M. A., 1960, Proc. Soc. Exp. Biol. Med. 104:388; Phan The Tran and Bender, M. A., 1961, in Radiobiology, Proceedings of the Third Australian Conference on Radiobiology, Ilbery, P. L. T., ed., Butterworth, London, p. 59). In a preferred embodiment, DMSO is used. DMSO, a liquid, is nontoxic to cells in low concentration. Being a small molecule, DMSO freely permeates the cell and protects intracellular organelles by combining with water to modify its freezability and prevent damage from ice formation. Adding plasma (for instance, to a concentration of 20-25%) can augment the protective effect of DMSO. After addition of DMSO, cells should be kept at 0° C. or below, since DMSO concentrations of about 1% may be toxic at temperatures above 4° C. My selected preferred cryopmotective agecnts are, in combination with TVEMF-expanded peripheral blood stem cells for the total composition: 20 to 40% dimethyl sulfoxide solution in 60 to 80% amino acid-glucose solution, or 15 to 25% hydroxyethyl starch solution, or 4 to 6% glycerol, 3 to 50% glucose, 6 to 10% dextran T10, or 15 to 25% polyethylene glycol or 75 to 850% amino acid-glucose solution. The amount of cryopreservative indicated above is preferably the total amount of cryopreservative in the entire composition (not just the amount of substance addcd to a composition).

While other substances, other than peripheral blood cells and a cryoprotective agent, may be present in a composition of the present invention to be cryopreserved, preferably cryopreservation of a TVEMF-expanded peripheral blood stem cell composition of the present invenvtion occurs with as few other substances as possible, for instance for reasons such as those discussed regarding the mechanism of freezing, above.

Preferably, a TVEMF-expanded peripheral blood stem cell composition of the present invention is cooled to a temperature in the range of about −120° C. to about −196° C., preferably about −130° C. to about −196° C., and even more preferably about 130° C. to about −150° C.

A controlled slow cooling rate is critical. Different cryoproteetive agents (Rapatz, G., et al., 1968, Cryobiology 5(1):18-25) and different cell types have different optimal cooling rates (see e.g. Rowe, A. W. and Rinfret, A. P., 1962, Blood 20:636; Rowe, A. W., 1966, Cryobiology 3(1):12-18; Lewis, J. P., et al., 1967, Transfusion 7(1):17-32; and Mazur, P., 1970, Science 168:939-949 for effects of cooling velocity on survival of peripheral cells (and on their transplantation potential)). The heat of fusion phase where water turns to ice should be minimal. The cooling procedure can be carried out by use of, e.g., a programmable freezing device or a methanol bath procedure.

Programmable freezing apparatuses allow determination of optimal cooling rates and facilitate standard reproducible cooling. Programmable controlled-rate freezers such as Cryomed or Planar permit tuning of the freezing regimen to the desired cooling rate curve. Other acceptable freezers may be, for example, Sanyo Modl MDF-1155ATN-152C and Model MDF-2136ATN-135C, Princeton CryoTech TEC 2000. For example, for peripheral blood cells or CD34+/CD38− cells in 10% DMSO and 20% plasma, the optimal rate is 1 to 3° C./minute from 0° C. to −200° C.

In a preferred embodiment, this cooling rate can be used for the cells of the invention. The cryogenic container holding the cells must be stable at cryogenic temperatures and allow for rapid heat transfcr for effective control of both freezing and thawing. Sealed plastic vials (e.g., Nunc, Wheaton cryules) or glass ampules can be used for multiple small amounts (1-2 ml), while larger volumes (100-200 ml) can be frozen in polyolefin bags (e.g., Delmed) held between metal plates for better heat transfer during cooling. (Bags of bone marrow cells have been successfully frozen by placing them in −80° C. freezers that, fortuitously, gives a cooling rate of approximately 3° C./minute).

In an alternative embodiment, the methanol bath method of cooling can be used. The methanol bath method is well suited to routine cryopreservation of multiple small items on a large scale. The method does not require manual control of the freezing rate nor a recorder to monitor the rate. In a preferred aspect, DMSO-treated cells are precooled on ice and transferred to a tray containing chilled methanol that is placed, in turn, in a mechanical refrigerator (e.g., Harris or Revco) at −130° C. Thermocouple measurements of the methanol bath and the samples indicate the desired cooling rate of 1 to 3°0 C./minute. After at least two hours, the specimens will reach a temperature of −80° C. and may be placed directly into liquid nitrogen (−196° C.) for permanent storage.

After thorough freezing, TVEMF-expanded stem cells can be rapidly transferred to a long-term cryogenic storage vessel (such as a freezer). In a preferred embodiment, the cells can be cryogenically stored in liquid nitrogen (−196° C.) or its vapor (−165° C). The storage temperature should be below −120° C., preferably below −130° C. Such storage is greatly facilitated by the availability of highly efficient liquid nitrogen refrigerators, which resemble large Thermos containers with an extremi-ely low vacuum and internal super insulation, such that heat leakage and nitrogen losses are kept to an absolute minimum.

The preferred apparatus and procedure for the cryopreservation of the cells is that manufactured by Thermogenesis Corp., Rancho Cordovo, Calif., utilizing their procedure for lowering the cell temperature to below −130° C. The cells are held in a Thermogenesis plasma bag during ficezing and storage.

Other freezers are commercially available. For instance, the “BioArchive” freezer not only freezes but also inventories a cryogenic sample such as blood or cells of the present invention, for instance managing up to 3,626 bags of frozen blood at a time. This freezer has a robotic arm that will retrieve a specific sample when instructed, ensuring that no other examples are disturbed or exposed to warmer temperatures. Other freezers commercially available include, but are not limited to, Sanyo Model MDF-1155 ATN-152C and Model MDF-2136 ATN-135C, and Princeton CryoTech TEC 2000.

After the temperature of the TVEMF-expanded peripheral blood stem cell composition is reduced to below −120°C., preferably below −130° C., they may be held in an apparatus such as a Thermogenesis freezer. Their temperature is maintained at a temperature of about −120° C. to −196° C., preferably −130° C. to −150° C. The temperature of a cryopreserved TVEMF-expanded peripheral blood stem cell composition of the present invention should not be above −120° C. for a prolonged period of time.

Cryopreserved TVEMF-expanded peripheral blood stem cells, or a composition thereof, according to the present invention may be frozen for an indefinite period of time, to be thawed when needed. For instance, a composition may be frozen for up to 18 years. Even longer time periods may work, perhaps even as long as the lifetime of the blood donor.

When needed, bags with the cells therein may be placed in a thawing system such as a Thermogenesis Plasma Thawer or other thawing apparatus such as in the Thermoline Thawer series. The temperature of the cryopreserved composition is raised to room temperature. In another preferred method of thawing cells mixed with a cryoprotective agent, bags having a cryopreserved TVEMF-expanded peripheral blood stem cell composition of the present invention, stored in liquid nitrogen, may be placed in the gas phase of liquid nitrogen for 15 minutes, exposed to ambient air room temperature for 5 minutes, and finally thawed in a 37° C. water bath as rapidily as possible. The contents of the thawed bags may be immediately diluted with an equal volume of a solution containintg 2.5% (weight/volume) human serum albumin and 5% (weight/volume) Dextran 40 (Solplex 40; Sifra, Verona, Italy) in isotonic salt solution and subsequently centrifuged at 400 g for ten minutes. The supernatant would be removed and the sedimenited cells resuspended in fresh albumin/Dextran solution. See Rubinstein, P. et al., Processing and cryopreservation of placental/umbilical cord blood for unrelated bone marrow reconstitution. Proc. Natl. Acad. Sci. 92:101 19-1012 (1995) for Removal of Hypertonic Cryoprotectant; a variation on this preferred method of thawing cells can be found in Lazzari, L. et al., Evaluation of the effect of cryopreservation on ex vivo expansion of hematopoietic progenitors from cord blood. Bone Morrow Trans. 28:693-698 (2001).

After the cells are raised in temperature to room temperature, they are available for research or regeneration therapy. The thawed TVEMF-expanded peripheral blood stem cell composition may be introduced directly into a mammal, preferably human, or used in its thawed form for instance for desired research. This solution in which the thawed cells are present may be completely washed away, and exchanged with another, or added to or otherwise manipulated as desired. Various additives may be added to the thawed compositions (or to a non-cryopreserved TVEMF-expanded peripheral blood stem cell composition) prior to introduction into a mammalian body, preferably soon to immediately prior to such introduction. Such additives include but are not limited to a growth factor, a copper chelating agent, a cytokine, a hormone, a suitable buffer or diluent. Preferably, G-CSF is added. Even more preferably, for humans, G-CSF is added in an amount of about 20 to about 40 micrograms/kg body weight, and even more preferably in an amount of about 30 micrograms/kg body weight. Also, prior to introduction, the TVEMF-expanded peripheral blood stem cell composition may be mixed with the mammal's own, or a suitable donor's, plasma, blood or albumin, or other materials that for instance may accompany blood transfusions. The thawed peripheral blood stem cells can be used for instance to test to see if there is an adverse reaction to a pharmaceutical that is desired to be used for treatment or they can be used for treatment.

While the FDA has not approved use of expanded peripheral blood stem cells for regeneration of tissue in the United States, such approval appears to be imminent. Direct injection of a sufficient amount of expanded peripheral blood stem cells should be able to be used to regenerate vital organs such as the heart, liver, pancreas, skin, muscle, gut, spleen, brain, and other tissues as mentioned throughout this application.

A TVEMF-expanded peripheral blood stem cell composition of the present invention should be introduced into a mammal, preferably a human, in an amount sufficient to achieve tissue repair or regeneration, or to treat a desired disease or condition. Preferably, at least 20 ml of a TVEMF-expanded peripheral blood stem cell composition having 10⁷ to 10⁹ stem cells per ml is used for any treatment, preferably all at once, in particular where a traumatic injury has occurred and immediate tissue repair needed. Thlis amount is particularly preferred in a 75-80 kg human. The amount of TVEMF-expanded peripheral blood stem cells in a composition being introduced into a mammal depends in part on the number of cells present in the source peripheral blood material (in particular if only a fairly limited amount is available). A preferred range of TVEMF-expanded peripheral blood stem cells introduced into a patient may be, for instance, about 10 ml to about 50 ml of a TVEMF-expanded peripheral blood stem cell composition having 10⁷ to about 10⁹ stem cells per ml, or potentially even more. While it is understood that a high concentration of any substance, administered to a mammal, may be toxic or even lethal, it is unlikely that introducing all of the TVEMF-expanded peripheral blood stem cells, for instance after TVEMF-expansion at least 7 times, will cause an overdose in TVEMF-expanded peripheral blood stem cells. Where peripheral blood from several donors or multiple collections from the same donor is used, the number of peripheral blood stem cells introduced into a mammal may be higher. Also, the dosage of TVEMF-cells that may be introduced to the patient is not limited by the amount of peripheral blood provided from collection from one individual; multiple administration, for instance once a day or twice a day, or once a week, or other administration time frames, may more easily be used. Also, where a tissue is to be treated, the type of tissue may warrant the use of as many TVEMF-expanded peripheral blood stem cells as are available, or the use of a smaller dose. For instance, liver may be easiest to treat and may require fewer stem cells than other tissues.

It is to be understood that, while the embodiment described above generally relates to cryopreserving TVEMF-expantded peripheral blood stem cells, TVEMF-expansion may occur after thawing of already cryopreserved, non-expanded, or non TVEMF-expanded, peripheral blood stem cells. Also, if cryopreservation is desired, TVEMF-expansion may occur both before and after freezing the cells. Blood banks, for instance, have cryopreserved compositions comprising peripheral blood stem cells in frozen storage, in case such is needed at some point in time. Such compositions may be thawed according to conventional methods and then TVEMF-expanded as described herein, including variations in the TVEMF-process as described herein. Thereafter, such TVEMF-expanded peripheral blood stem cells are considered to be compositions of the present invention, as described above. TVEMF-expansion prior to cryopreserving is preferred, for instance as if a traumatic injury occurs, a patient's peripheral blood stem cells have already been expanded and do not require precious extra days to prepare.

Also, while not preferred, it should be noted that TVEMF-expanded peripheral blood stem cells of the present invention may be cryopreserved, and then thawed, and then if not used, cryopreserved again. Prior to the cells being frozen, are preferably TVEMF-expanded (that is, increased in number, not size). The cells may also be expanded after being frozen and then thawed, even if already expanded before freezing.

Expansion of peripheral blood stem cells may take several days. In a situation where it is important to have an immediate supply of peripheral blood stem cells, such as a life-or-death situation or in the case of a traumatic injury, especially if research needs to be accomplished prior to reintroduction of the cells, several clays may not be available to await the expansion of the peripheral blood stein cells. It is particularly desirable, therefore, to have such expanded peripheral blood stem cells available from birth forward in anticipation of an emergency where every minute in delaying treatment can mean the difference in life or death.

Also, it is to be understood that the TVEMF-expanded peripheral blood stem cells of the present application may be introduced into a mammal, preferably the source mammal (mammal that is the source of the peripheral blood), after TVEMF-expansion, with or without cryopreservation. However, such introduction need not be limited to only the source mammal (autologous); the TVEMF-expanded cells may also be transferred to a different mammal (allogenic).

Also, it is to be understood that, while peripheral blood is the preferred source of adult stem cells for the present invention, adult stem cells from bone marrow may also be TVEMIF-expanded and used in a manner similar to peripheral blood stem cells in the present invention. Bone marrow is not a readily available source of stem cells, but must be collected via apheresis or some other expensive and painful method.

The present invention may also include a method of researching a disease state comprising introducing a TVEMF-expanded stem cell into a test system for the disease state. Such as system may include, but is not limited to, for instance a mammal having the disease, an appropriate animal model for studying the disease or an in vitro test system for studying the disease. TVEMF-expanded peripheral blood stem cells may be used for research for possible cures for the following diseases:
I. Diseases resulting from a failure or dysfunction of normal blood cell production and maturation, hyperproliferative stem cell disorders, aplastic anemia, pancytopenia, thrombocytopenia, red cell aplasia, Blackfan-Diamond syndrome due to drugs, radiation, or infection, idiopathic;
II. Hermatopoietic malignancies, acute lymphoblastic (lymphocytic) leukemia, chronic lymphocytic leukemia, acute myelogenous leukemia, chronic myelogenious leukemia, acute malignant myelosclerosis, multiple mycloma, polycythemia vera, agnogenic myelometaplasia, Waldenstrom's macroglobulinemia, Hodgkin's lymphoma, non-Hodgkins's lymphoma;
III. Immunosuppression in patients with malignant, solid tumors, malignant melanoma, carcinoma of the stomach, ovarian carcinoma, breast carcinoma, small cell lung, carcinoma, retinoblastom a, testicular carcinoma, glioblastoma, rhabdomyosarcoma, neuroblastomia, Ewing's sarcoma, lymphoma;
IV. Autoimmune diseases, rheumatoid arthritis, diabetes type I chronic hepatitis, multiple sclerosis, and systemic lupus erythematosus;
V. Genetic (congenital) disorders, anemias, familial aplastic, Fanconi's syndrome, Bloom's syndrome, pure red cell aplasia (PRCA), dyskeratosis congenital, Blackfan-Diamond syndrome, congenital dyserythropoietic syndromes I-IV, Chwachmann-Diamond syndrome, dihydrofolate reductase deficiencies, formamino transferase deficiency, Lesch-Nyhan syndrome, congenital spherocytosis, congenital elliptocytosis, congenital stomatocytosis, congenital Rh null disease, paroxysmal nocturnal hemoglobinuria, G6PD (glucose-6-phosphate dehydrogenase), variants 1,2,3, pyruvate kinase deficiency, congenital erythropoietin sensitivity, deficiency, sickle cell disease and trait, thalassemia alpha, beta, gamma met-hemoglobinemia, congenital disorders of immunity, severe combined immunodeficiency disease, (SCID), bare lymphocyte syndrome, ionophore-responsive combined, immunodeficiency, combined immunodeficiency with a capping abnormality, nucleoside phosphorylase deficiency, granulocyte actin deficiency, infantile agranulocytosis, Gaucher's disease, adenosine deaminase deficiency, Kostmann's syndrome, reticular dysgenesis, congenital leukocyte dysfunction syndromes; and
VI. Others including osteopetrosis, myelosclerosis, acquired hemolytic anemias, acquired immunodeficiencies, infectious disorders causing primary or secondary immunodeficiencies, bacterial infections (e.g., Brucellosis, Listerosis, tuberculosis, leprosy), parasitic infections (e.g., malaria, Leishmaniasis), fungal infections, disorders involving disproportions in lymphoid cell sets and impaired immuune functions due to aging phagocyte disorders, Kostmann's agranulocytosis, chronic granulomatous disease, Chediak-Higachi syndrome, neutrophil actin deficiency, neutrophil membrane GP-180 deficiency, metabolic storage diseases, mucopolysaccharidoses, mucolipidoses, miscellaneous disorders involving immune mechanisms, Wiskott-Aldrich Syndrome, alpha 1-antitrypsin deficiency.

During the entire process of expansion, preservation, and thawing, peripheral blood stem cells of the present invention maintain their three-dimensional geometry and their cell-to-cell support and cell-to-cell geometry.

While preferred embodiments have been herein described, those skilled in the art will understand the present invention to include various changes and modifications. The scope of the invention is not intended to be limited to the above-described embodiments.

I claim:

1. A process for preparing a peripheral blood stem cell composition comprising the steps of:
a. placing a peripheral blood mixture in a culture chamber of a TVEMF-bioreactor; and
b. subjecting the peripheral blood mixture to a TVEMF and TVEMF-expanding the peripheral blood stem cells in the TVEMF-bioreactor to prepare the peripheral blood stem cell composition.

2. The process according to claim 1, wherein said TVEMF is about 0.05 to about 6.0 gauss.

3. The process according to claim 1, wherein said TVEMF-expanding continues until the number per volume of TVEMF-expanded peripheral blood stem cells is more than 7 times the number per volume of peripheral blood stem cells placed in the TVEMF-bioreactor.

4. The process according to claim 1, further comprising collecting peripheral blood prior to placing the peripheral blood mixture in a TVEMF-bioreactor.

5. The process of claim 4, wherein said peripheral blood is human peripheral blood.

6. The process according to claim 1, further comprising collecting thawed cryopreserved peripheral blood from a peripheral blood storage facility prior to adding the peripheral blood to the peripheral blood mixture.

7. The process of claim 1, further comprising a step of removing toxic material from the peripheral blood mixture prior to TVEMF-expansion.

8. The process of claim 1, wherein the TVEMF-bioreactor has an integral TVEMF source.

9. The process of claim 1, wherein the TVEMF-bioreactor has an adjacent TVEMF source.

10. The process of claim 1, wherein the peripheral blood mixture comprises CD34+/CD38− peripheral blood stem cells separated from other peripheral blood components.

11. The process of claim 1, wherein the peripheral blood mixture comprises a buffy coat separated from other peripheral blood components.

12. The process of claim 1, wherein the peripheral blood mixture comprises peripheral blood free of red blood cells.

13. The process of claim 1, further comprising the steps of transferring the TVEMF-expanding cells of the pheripheral blood stem cell composition into a cryogenic container having a temperature, and lowering the temperature of the cryogenic container to a temperature of from −120° C. to−196° C. at a controlled rate.

14. The process of claim 13, further comprising a step of removing toxic material from the peripheral blood stem cell composition prior to lowering the temperature to a temperature of from −120° C. to −196° C. at a controlled rate.

15. The process of claim 13, further comprising, after the step of lowering the temperature, a step of maintaining the temperature of the cryogenic container to a temperature of from −120° C. to −196° C., for a period of time.

16. The process of claim 15, wherein said period of time is at least 1 year.

17. The process of claim 15, further comprising, after said lowering and maintaining of temperature, a step of increasing the temperature of the cryogenic container at a controlled rate to a temperature suitable for introducing the peripheral blood stem cell composition to a mammal.

18. The process of claim 17, wherein toxic material has been removed from said increased temperature peripheral blood stem cell composition.

19. The process of claim 13, further comprising the step of adding a cryopreservative to the TVEMF-expanded cells of the peripheral blood stem cell composition before the step of lowering the temperature.
